(19) United States
(12) Patent Application Publication
(10) Pub. No.: US 2010/0100082 A1
(43) Pub. Date: Apr. 22, 2010

(54) SURGICAL OPERATION APPARATUS

(75) Inventors: Koh SHIMIZU, Hachioji-shi (JP); Naoko TAHARA, Hachioji-shi (JP)

Correspondence Address: OSTROLENK FABER GERB & SOFFEN, 1180 AVENUE OF THE AMERICAS, NEW YORK, NY, 100368403, US

(73) Assignee: Olympus Medical Systems Corporation

(21) Appl. No.: 12/644,617

(22) Filed: Dec. 22, 2009

Related U.S. Application Data
(63) Continuation of application No. 11/444,012, filed on May 31, 2006.

(30) Foreign Application Priority Data
May 31, 2005 (JP) 2005-160623

(51) Int. Cl. A61B 19/00

(52) U.S. Cl. 606/1

(57) ABSTRACT

A surgical operation apparatus comprises an operation apparatus main body having a control section, a plurality of switches which is provided in the operation apparatus main body and operated by a user, a switch detection circuit which is connected to the control section and also connected to the switches and which detects the switch operable by the user among the switches, light emitting units which are provided in the switches themselves or on the periphery of the switches and which light the switches, and a light emitting circuit which is connected to the control section and also connected to the light emitting units and which causes the light emitting unit of the operable switch among the switches to emit light on the basis of a signal from the switch detection circuit.

### CROSS-REFERENCE TO RELATED APPLICATIONS

This application is a continuation of prior application U.S. Ser. No. 11/444,012, filed May 31, 2006, which claims the benefit of priority from prior Japanese Patent Application No. 2005-160623, filed May 31, 2005, the entire contents of which are incorporated herein by reference.

### BACKGROUND OF THE INVENTION

1. Field of the Invention

This invention relates to a surgical operation apparatus used, for example, to supply electric power to various devices during a surgical operation.

2. Description of the Related Art

Jpn. Pat. Appln. KOKAI Publication No. 2002-233537 has disclosed an endoscopic system capable of efficiently and reliably operating a plurality of medical devices with one remote control. In this system, the remote control comprises a plurality of function switches, a plurality of device name display units having electroluminescent panels and character transmitting sections, and a plurality of function setting units. Further, this system can control the light emission of the electroluminescent panel by a CPU so that the device name display unit of a device to be used alone emits light. This ensures that the display of the remote control can be visually recognized even in an operating room where it is relatively dark except for an operated part of a patient.

Jpn. Pat. Appln. KOKAI Publication No. 2001-314411 has disclosed a surgical operation system with a good manipulability which allows treatment with a handpiece actually gripped by an operator among a plurality of handpieces. This system comprises sensors provided in grip portions of the plurality of handpieces, and an output switch unit which switches outputs of drive signals to a handpiece connection portion on the basis of detection signals of the sensors. In this system, an LED in the vicinity of a selection switch of a remote switch emits light after manipulation, or an LED provided in the vicinity of a selection switch on an operation panel of an apparatus main body emits light, such that a selection result (manipulation result) can be reported.

Jpn. Pat. Appln. KOKAI Publication No. 2001-95748 has disclosed a connected device recognition system, and this system recognizes, on the device side, which device is connected to which terminal by which cable or cord when a plurality of devices is used, thereby making it possible to easily check a wiring state.

This system comprises a system controller which recognizes the connection of the devices and which reports a connection state. The system controller can select any data during an operation and display it on a central display panel beside a monitor. This central display panel comprises a display function to display the state of the devices and to display operation switches and the like as setting screens, and an operation function in which a predetermined area of a touch sensor is touched to perform an operation with the operation switches.

### BRIEF SUMMARY OF THE INVENTION

A surgical operation apparatus according to one aspect of the present invention comprises: an operation apparatus main body having a control section; a plurality of switches which is provided in the operation apparatus main body and operated by a user; a switch detection circuit which is connected to the control section and also connected to the switches and which detects the switch operable by the user among the switches; light emitting units which are provided in the switches themselves or on the periphery of the switches and which light the switches; and a light emitting circuit which is connected to the control section and also connected to the light emitting units and which causes the light emitting unit of the operable switch among the switches to emit light on the basis of a signal from the switch detection circuit.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing the configuration of a surgical operation system according to a first embodiment of the present invention;

FIG. 2 is a schematic perspective view showing an ultrasonic vibration driver in the surgical operation system according to the first embodiment;

FIG. 3 is a schematic block diagram showing the internal configuration of the ultrasonic vibration driver in the surgical operation system according to the first embodiment and showing how the ultrasonic vibration driver is connected with peripheral devices;

FIG. 4 is a schematic diagram to explain that a first handpiece and a second handpiece are interchangeably attached to the surgical operation system according to the first embodiment;

FIG. 5A shows a front view of the ultrasonic vibration driver in the surgical operation system according to the first embodiment, and this schematic view shows a situation where a light emitting unit on the outer periphery of an output set value up switch is emitting light while a light emitting unit on the outer periphery of an output set value down switch is lit out;

FIG. 5B shows a front view of the ultrasonic vibration driver in the surgical operation system according to the first embodiment, and this schematic view shows a situation where both of the light emitting units on the outer periphery of the output set value up switch and the output set value down switch are emitting light;

FIG. 5C shows a front view of the ultrasonic vibration driver in the surgical operation system according to the first embodiment, and this schematic view shows a situation where the light emitting unit on the outer periphery of the output set value up switch is lit out while the light emitting unit on the outer periphery of the output set value down switch is emitting light;

FIG. 6A is a schematic front view showing the ultrasonic vibration driver in the surgical operation system according to a fourth embodiment of the present invention, wherein the output set value up switch and the output set value down switch are lit out;

FIG. 6B is a schematic sectional view showing the ultrasonic vibration driver in the surgical operation system according to the fourth embodiment of the present invention, wherein the output set value up switch and the output set value down switch are lit out;

FIG. 6C is a schematic front view showing the ultrasonic vibration driver in the surgical operation system according to the fourth embodiment of the present invention, wherein the output set value up switch and the output set value down switch are lit;

FIG. 6D is a schematic sectional view showing the ultrasonic vibration driver in the surgical operation system according to the fourth embodiment of the present invention, wherein the output set value up switch and the output set value down switch are lit out;

FIG. 7A shows a front view of the ultrasonic vibration driver in the surgical operation system according to the fourth embodiment of the present invention, and this schematic view shows a situation where the output set value up switch is emitting light while the output set value down switch is lit out;

FIG. 7B shows a front view of the ultrasonic vibration driver in the surgical operation system according to the fourth embodiment of the present invention, and this schematic view shows a situation where both of the output set value up switch and the output set value down switch are emitting light;

FIG. 7C shows a front view of the ultrasonic vibration driver in the surgical operation system according to the fourth embodiment of the present invention, and this schematic view shows a situation where the output set value up switch is lit out while the output set value down switch is emitting light;

FIG. 8A shows a front view of the ultrasonic vibration driver in the surgical operation system according to a fifth embodiment of the present invention, and this schematic view shows a situation where the output set value up switch is operable while the output set value down switch is inoperable;

FIG. 8B shows a front view of the ultrasonic vibration driver in the surgical operation system according to the fifth embodiment of the present invention, and this schematic view shows a situation where both of the output set value up switch and the output set value down switch are operable;

FIG. 8C shows a front view of the ultrasonic vibration driver in the surgical operation system according to the fifth embodiment of the present invention, and this schematic view shows a situation where the output set value up switch is inoperable while the output set value down switch is operable;

FIG. 9 is a schematic diagram showing the configuration of the surgical operation system according to sixth to eighth embodiments of the present invention;

FIG. 10A shows a front view of the ultrasonic vibration driver in the surgical operation system according to the sixth to eighth embodiments, and this schematic view shows a situation where first to third output mode switches are selectable;

FIG. 10B shows a front view of the ultrasonic vibration driver in the surgical operation system according to the sixth to eighth embodiments, and this schematic view shows a situation where the first output mode switch is selected; and

FIG. 10C shows a front view of the ultrasonic vibration driver in the surgical operation system according to the sixth to eighth embodiments, and this schematic view shows a situation where the second output mode switch is selected.

### DETAILED DESCRIPTION OF THE INVENTION

A best mode for carrying out this invention will hereinafter be described with reference to the drawings.

A first embodiment will be described with FIGS. 1 to 5C.

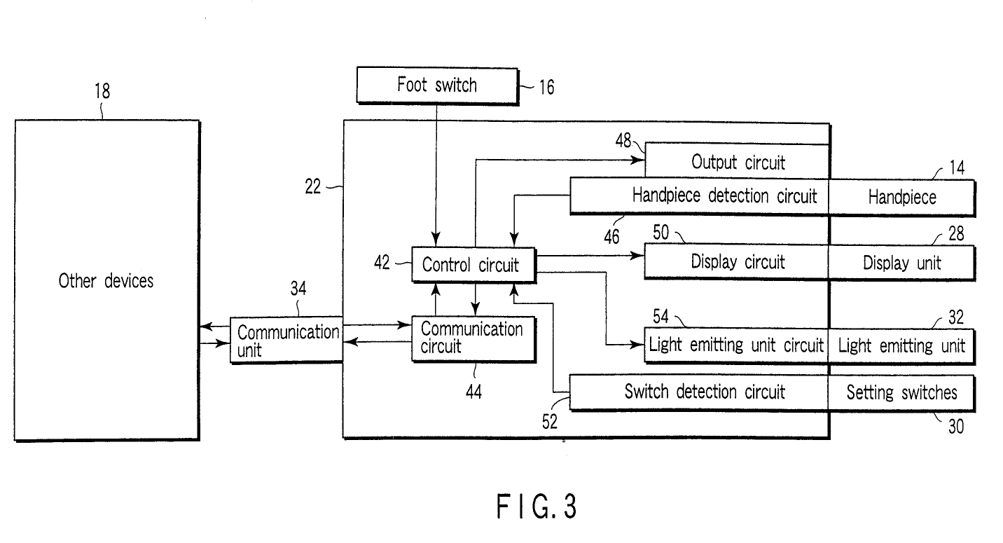

As shown in FIG. 1, a surgical operation system 10 comprises an ultrasonic vibration driver 12 which is a surgical operation apparatus, a handpiece 14 which is an instrument for an operator to perform an ultrasonic treatment, and a foot switch 16 for control over whether or not to generate ultrasonic vibration from the handpiece 14. Among the above, the ultrasonic vibration driver 12 can also be used as a power supply unit for other devices 18 (see FIG. 3). It is to be noted that, although not shown in the drawings, the other devices 18 include a high-frequency current output device, a water carrier, an air carrier, etc.

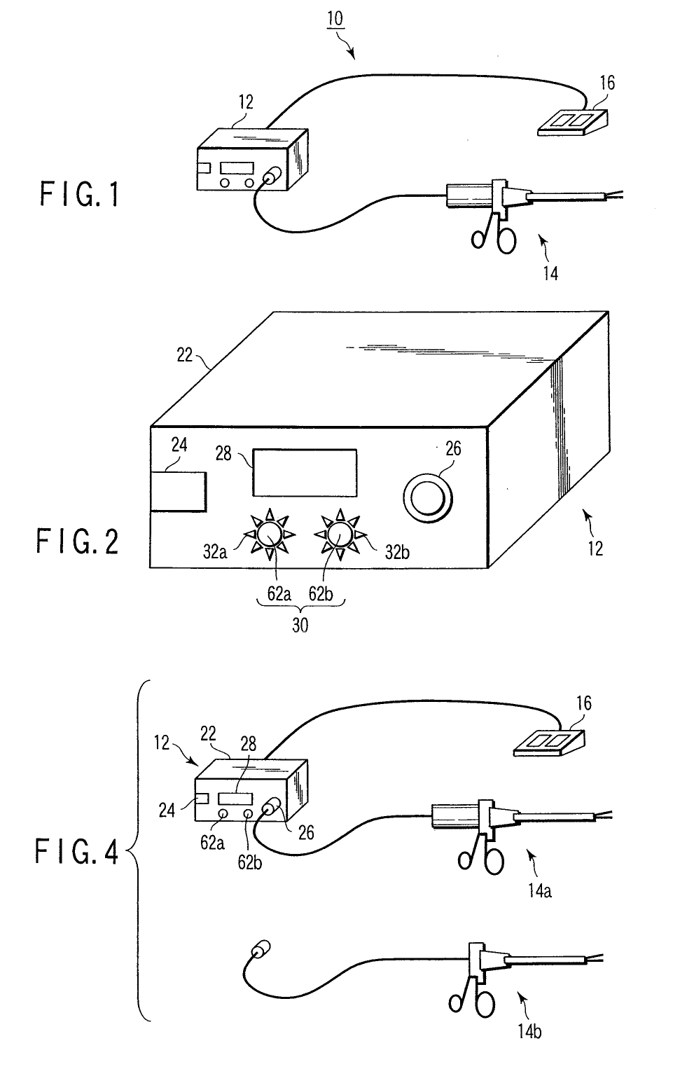

As shown in FIG. 2, the ultrasonic vibration driver 12 comprises a main body 22, a power switch 24, a handpiece connection portion 26, a display unit 28, various setting switches 30, light emitting units (light emitting means) 32 and a communication unit 34 (see FIG. 3). The power switch 24, the handpiece connection portion 26, the display unit 28, the various setting switches 30, the light emitting units 32 and the communication unit 34 are disposed in the main body 22.

For the display unit 28, a liquid crystal display, an organic electroluminescent display, a 7-segment light emitting diode (LED) or the like is used. For the various setting switches 30, press switches, pressure switches, optical switches and the like are used. For the light emitting units 32, LEDs or the like are used. The light emitting units 32 are disposed in the various setting switches 30 or on the periphery of the various setting switches 30. When the light emitting units 32 are disposed in the various setting switches 30, the various setting switches 30 are transparent or semitransparent.

As shown in FIG. 3, inside the main body 22 of the ultrasonic vibration driver 12, there are arranged a control circuit 42, a communication circuit 44 which is connected with the other devices 18 through the communication unit 34, a handpiece detection circuit 46, an output circuit 48, a display circuit 50, a switch detection circuit (switch recognition means) 52 and a light emitting unit circuit (light emitting means) 54.

The foot switch 16 (see FIG. 1), the communication circuit 44, the handpiece detection circuit 46, the output circuit 48, the display circuit 50, the switch detection circuit 52 and the light emitting unit circuit 54 are connected to the control circuit 42. Thus, on receipt of a signal sent from the foot switch 16, signals of the other devices 18 sent from the communication circuit 44, a signal sent from the handpiece detection circuit 46 concerning the kind (model, type, etc.) of the handpiece 14 connected to the handpiece detection circuit 46, and a switch signal sent from the switch detection circuit 52, the control circuit 42 transmits signals to the communication circuit 44, the display circuit 50 and the light emitting unit circuit 54.

The display circuit 50 and the light emitting unit circuit 54 comprise liquid crystal display drivers, LED drivers, etc. On receipt of the signals from the control circuit 42, the display circuit 50 and the light emitting unit circuit 54 drive the liquid crystal display, the organic electroluminescent display, the 7-segment LED and the LED. The liquid crystal display, the organic electroluminescent display, the 7-segment LED and the LED are capable of single color light emission (black-and-white display), multiple color light emission (color display), changing colors and flashing.

As shown in FIG. 2, the various setting switches 30 of the ultrasonic vibration driver 12 comprise an output set value up switch 62a and an output set value down switch 62b. A light emitting unit 32a is provided on the periphery of the output set value up switch 62a. A light emitting unit 32b is provided on the periphery of the output set value down switch 62b.

As shown in FIG. 4, a first handpiece 14a is connected to the handpiece connection portion 26 of the ultrasonic vibration driver 12. In a situation where the first handpiece 14a is connected, it is possible to change an output set value of the ultrasonic vibration driver 12. Further, the control circuit 42 and the output circuit 48 of the ultrasonic vibration driver 12 are set so that the second handpiece 14b cannot generate ultrasonic vibration in a situation where a second handpiece 14b whose specification such as model or type is different from that of the first handpiece 14a is connected to the handpiece connection portion 26.

Next, the function of the surgical operation system 10 according to this embodiment will be described.

The power switch 24 shown in FIG. 2 is switched on (the power switch 24 is turned on). When the handpiece detection circuit 46 detects that the first handpiece 14a is connected to the handpiece connection portion 26, the handpiece detection circuit 46 transmits the detection signal to the control circuit 42. The control circuit 42 transmits to the output circuit 48 a signal indicating that the output circuit 48 may output to the first handpiece 14a. The output circuit 48 prepares to cause the first handpiece 14a to generate the ultrasonic vibration. At this moment, the control circuit 42 transmits a signal from the switch detection circuit 52 to the display circuit 50, and displays on the display unit 28 a current set value of the output to the first handpiece 14a (not shown).

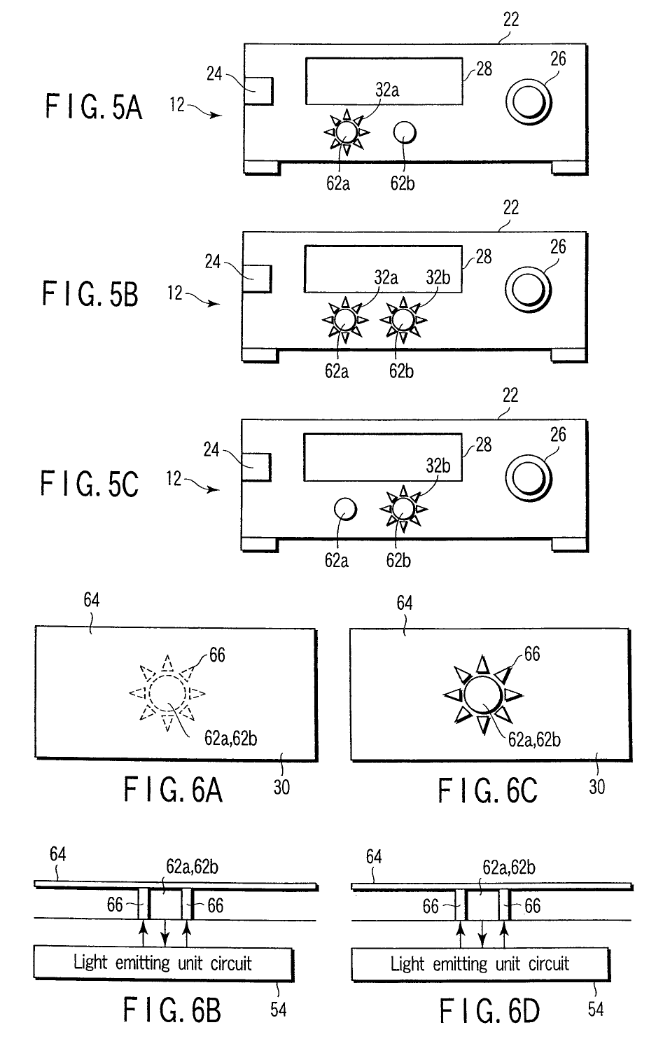

In a situation where the power switch 24 is turned on, the output set value of the ultrasonic vibration driver 12 is at the minimum value. At this moment, since the output set value of the ultrasonic vibration driver 12 is at the minimum value, it is not possible to further decrease the output set value. Therefore, the output set value up switch 62a alone is operable of the output set value up switch 62a and the output set value down switch 62b shown in FIG. 2. Thus, as shown in FIG. 5A, the light emitting unit 32a on the periphery of the output set value up switch 62a alone is lit, while the light emitting unit 32b on the periphery of the output set value down switch 62b is continuously lit out. That is, the light emitting unit 32a on the periphery of the operable switch 62a alone is lit.

From this state, the output set value up switch 62a of the ultrasonic vibration driver 12 is operated to increase the output set value. At this moment, the output set value can be decreased again or can be further increased. Therefore, both of the output set value up switch 62a and the output set value down switch 62b are operable. Thus, as shown in FIG. 5B, both the light emitting unit 32a on the periphery of the output set value up switch 62a and the light emitting unit 32b on the periphery of the output set value down switch 62b are lit. That is, the output set value up switch 62a stays lit, while the light emitting unit 32b of the output set value down switch 62b is newly lit. At this moment, the display unit 28 displays a state in which the output set value has increased from the minimum state.

Furthermore, the output set value of the ultrasonic vibration driver 12 is increased from this state, and the output set value is set to the maximum value. At this moment, since the output set value of the ultrasonic vibration driver 12 is at the maximum value, the output set value cannot be further increased. Therefore, as shown in FIG. 5C, the output set value down switch 62b alone is operable of the output set value up switch 62a and the output set value down switch 62b. Thus, the light emitting unit 32b on the periphery of the output set value down switch 62b alone stays lit, while the light emitting unit 32a on the periphery of the output set value up switch 62a is lit out. At this moment, the display unit 28 displays a state in which the output set value is at the maximum.

When the first handpiece 14a is used, the switch that is lit is operated of the output set value up switch 62a and the output set value down switch 62b in order to set the output set value. At this moment, a user checks the output set value with the display unit 28, and performs the operation described above to set a desired output set value.

The user holds the first handpiece 14a, and presses the foot switch 16 connected to the ultrasonic vibration driver 12 to transmit a corresponding signal to the control circuit 42. The control circuit 42 transmits to the output circuit 48 a signal to be output by the output circuit 48. The output circuit 48 which has prepared for outputting passes a current to the first handpiece 14a to actuate the ultrasonic vibration. The first handpiece 14a generates ultrasonic output oscillation to perform various treatments on a patient or the like.

When the pressing of the foot switch 16 is released, a corresponding signal is transmitted to the control circuit 42. The control circuit 42 transmits to the output circuit 48 a signal to stop the output. Thus, the current running from the output circuit 48 to the first handpiece 14a stops. Therefore, the ultrasonic output oscillation from the first handpiece 14a stops.

Next, a case will be described wherein the first handpiece 14a is replaced with the second handpiece 14b.

In this case, the first handpiece 14a is removed from the handpiece connection portion 26, and the second handpiece 14b is connected to the handpiece connection portion 26. Since the second handpiece 14b is connected to the handpiece connection portion 26 instead of the first handpiece 14a, the output set value of the ultrasonic vibration driver 12 cannot be changed.

Here, the handpiece detection circuit 46 detects the kind of the handpiece, and transmits a corresponding signal to the control circuit 42. Then, the signal is transmitted from the control circuit 42 to the output circuit 48. At this moment, since the handpiece detection circuit 46 has detected that the second handpiece 14b is connected to the handpiece connection portion 26, the control circuit 42 transmits to the output circuit 48 a signal indicating not to provide any output from the output circuit 48 to drive ultrasonic waves.

Therefore, in this state, the output set value up switch 62a and the output set value down switch 62b cannot be operated. Even if they are operated, such an operation cannot change the output set value up switch 62a and the output circuit 48. Thus, the output set value up switch 62a and the output set value down switch 62b are continuously lit out.

The other devices 18 and the control circuit 42 bidirectionally transmit signals to each other via the communication unit 34 and the communication circuit 44, thereby performing a desired operation.

As described above, the following can be said according to this embodiment.

When the ultrasonic vibration driver 12 is operated, it is possible to readily recognize the operable (workable) switch in accordance with whether the light emitting units 32a and 32b on the periphery of the switches 62a and 62b are lighting or lighting out. Therefore, the user can efficiently operate the devices such as the ultrasonic vibration driver 12.

Since the switch which is lit out cannot be operated (cannot be actuated) even if an attempt is made, the user can readily recognize, for example, an error in operation of the ultrasonic vibration driver 12.

Therefore, in a situation where a plurality of devices and various handpieces are connected to the ultrasonic vibration driver 12, the operable switch is clearly indicated before the user operates the ultrasonic vibration driver 12, such that the user can, for example, efficiently set the devices.

In addition, it is also preferable that the light emitting units 32a and 32b are provided only on the upper or lower side of the switches 62a and 62b rather than on the periphery of the switches 62a and 62b.

Next, a second embodiment will be described. This embodiment is a modification of the first embodiment, so that the same signs are assigned to the same members as those described in the first embodiment and those members are not described in detail.

In this embodiment, the light emitting units (indication lights) 32a and 32b which change emitted light are provided on the periphery of the output set value up switch 62a and the output set value down switch 62b of the ultrasonic vibration driver 12, respectively. The light emitting units 32a and 32b can preferably emit light of different colors when the power switch 24 is turned on. Thus, the light emitting units 32a and 32b preferably comprise light emitters such as LEDs with, for example, two kinds of colors: one used when the power switch 24 is turned on and one different from the former.

Next, the function of the surgical operation system 10 according to this embodiment will be described.

The power switch 24 is switched on (the power switch 24 is turned on). At this moment, the output set value up switch 62a and the output set value down switch 62b keep lighting. In a situation where the power switch 24 is turned on, the output set value of the ultrasonic vibration driver 12 is at the minimum value. At this moment, since the output set value of the ultrasonic vibration driver 12 is at the minimum value, it is not possible to further decrease the output set value. Therefore, the output set value up switch 62a alone is operable of the output set value up switch 62a and the output set value down switch 62b. Thus, the color of the light emitted by the output set value up switch 62a changes, while the output set value down switch 62b has been lighting since the power switch 24 was turned on. That is, the lighting state of the operable switch alone changes when the power switch 24 is turned on after the output set value of the ultrasonic vibration driver 12 is recognized.

From this state, the output set value up switch 62a of the ultrasonic vibration driver 12 is operated to increase the output set value. At this moment, the output set value can be decreased again or can be further increased. Therefore, both of the output set value up switch 62a and the output set value down switch 62b are operable. Thus, the lighting state of both the output set value up switch 62a and the output set value down switch 62b are changed when the power switch 24 is turned on. That is, the output set value up switch 62a stays the lighting state changing, while the output set value down switch 62b newly changes the lighting state.

From this state, the output set value of the ultrasonic vibration driver 12 is increased to set the output set value at the maximum value. At this moment, since the output set value of the ultrasonic vibration driver 12 is at the maximum value, the output set value cannot be further increased. Therefore, the output set value down switch 62b alone is operable of the output set value up switch 62a and the output set value down switch 62b. Thus, the output set value down switch 62b alone stays the lighting state changing, while the output set value up switch 62a changes to the lighting state exhibited when the power switch 24 is turned on.

When the first handpiece 14a is used, the switch whose lighting state is changing is operated of the output set value up switch 62a and the output set value down switch 62b, thereby setting the output set value.

Next, the power switch 24 is switched off. In this state, the first handpiece 14a is removed from the handpiece connection portion 26, and the second handpiece 14b is connected to the handpiece connection portion 26. At this moment, since the second handpiece 14b is connected to the handpiece connection portion 26 instead of the first handpiece 14a, the output set value of the ultrasonic vibration driver 12 cannot be changed. Therefore, in this state, the output set value up switch 62a and the output set value down switch 62b cannot be operated. Even if they are operated, such an operation cannot change the output set value up switch 62a and the output set value down switch 62b. Thus, the output set value up switch 62a and the output set value down switch 62b stay the lighting state exhibited when the power switch 24 is turned on.

Next, a third embodiment will be described. This embodiment is a modification of the first and second embodiments, so that the same signs are assigned to the same members as those described in the first and second embodiments and those members are not described in detail.

In this embodiment, the output set value up switch 62a and the output set value down switch 62b of the ultrasonic vibration driver 12 are provided with the light emitting units (indication lights) 32a and 32b which generate and flash colored lights in a lighting state when the power switch 24 is turned on. The light emitting units 32a and 32b can preferably emit light of different colors when the power switch 24 is turned on. Thus, the light emitting units 32a and 32b preferably comprise light emitters such as LEDs with, for example, two kinds of colors: one used when the power switch 24 is turned on and one different from the former.

Next, the function of the surgical operation system 10 according to this embodiment will be described.

The power switch 24 is switched on (the power switch 24 is turned on). At this moment, the output set value up switch 62a and the output set value down switch 62b always keep lighting. In a situation where the power switch 24 is turned on, the output set value of the ultrasonic vibration driver 12 is at the minimum value. At this moment, since the output set value of the ultrasonic vibration driver 12 is at the minimum value, it is not possible to further decrease the output set value. Therefore, the output set value up switch 62a alone is operable of the output set value up switch 62a and the output set value down switch 62b. Thus, the output set value up switch 62a alone changes the color of the emitted light and flashes, while the output set value down switch 62b has been lighting since the power switch 24 was turned on. That is, the lighting state of the operable switch alone changes to a color-changing and flashing state. In other words, another light emitter different from the one used when the power switch 24 is turned on flashes.

From this state, the output set value up switch 62a of the ultrasonic vibration driver 12 is operated to increase the output set value. At this moment, the output set value can be decreased again or can be further increased. Therefore, both of the output set value up switch 62a and the output set value down switch 62b are operable. Thus, the lighting state of both the output set value up switch 62a and the output set value down switch 62b are changed when the power switch 24 is turned on. That is, the output set value up switch 62a keeps the lighting state changing, while the output set value down switch 62b newly changes the lighting state. Thus, the output set value down switch 62b changes the color of the emitted light and flashes.

Furthermore, from this state, the output set value of the ultrasonic vibration driver 12 is increased to set the output set value at the maximum value. At this moment, since the output set value of the ultrasonic vibration driver 12 is at the maximum value, the output set value cannot be further increased. Therefore, the output set value down switch 62b alone is operable of the output set value up switch 62a and the output set value down switch 62b. Thus, the output set value down switch 62b alone keeps the lighting state changing and flashing, while the output set value up switch 62a changes to the lighting state exhibited when the power switch 24 is turned on. Thus, the output set value up switch 62a stays lit.

When the first handpiece 14a is used, the switch whose lighting state is changing and which is flashing is operated of the output set value up switch 62a and the output set value down switch 62b, thereby setting the output set value.

Next, the first handpiece 14a is removed from the handpiece connection portion 26, and the second handpiece 14b is connected to the handpiece connection portion 26. At this moment, since the second handpiece 14b is connected to the handpiece connection portion 26 instead of the first handpiece 14a, the output set value of the ultrasonic vibration driver 12 cannot be changed. Therefore, in this state, the output set value up switch 62a and the output set value down switch 62b cannot be operated. Even if they are operated, such an operation cannot change the output set value up switch 62a and the output set value down switch 62b. Thus, the output set value up switch 62a and the output set value down switch 62b keep the lighting state exhibited when the power switch 24 is turned on.

It is to be noted that the light emitting unit 32a, 32b comprises the two kinds of light emitters in the description of the present embodiment. However, it is also preferable that the light emitting unit comprise only one kind of light emitter, and that the light emitter can be changed into a lighting and flashing state so that the user may recognize the operable switch.

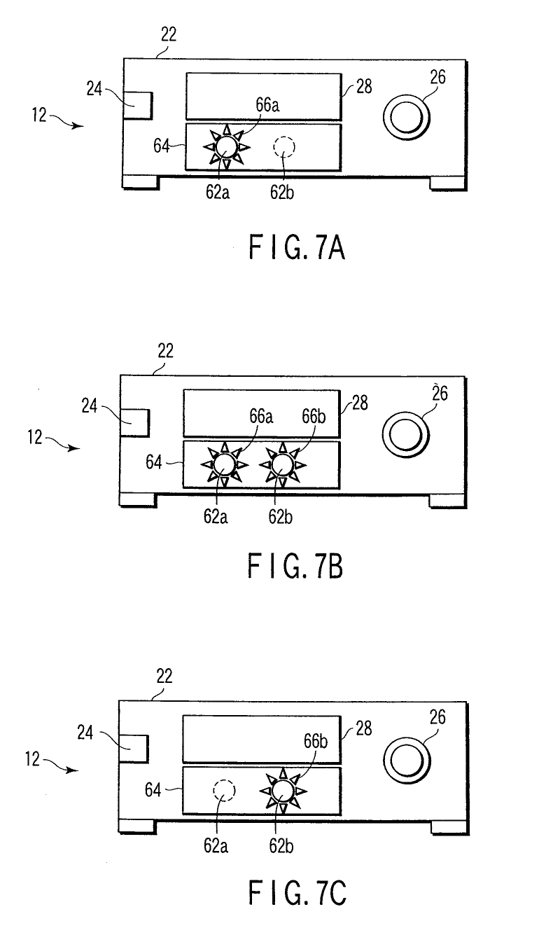

Next, a fourth embodiment will be described with FIGS. 6A to 7C. This embodiment is a modification of the first embodiment, so that the same signs are assigned to the same members as those described in the first embodiment and those members are not described in detail.

In this embodiment, the setting switches 30 of the ultrasonic vibration driver 12 comprise the output set value up switch 62a and the output set value down switch 62b. The surface of the various setting switches 30 is covered with a semitransparent soft cover 64.

FIGS. 6A to 6D show a switch unit when the surface of the various setting switches 30 is covered with the semitransparent soft cover 64. When the output set value up switch 62a and the output set value down switch 62b are lit out, the output set value up switch 62a and the output set value down switch 62b can be visually recognized only at positions where the user is close thereto.

As shown in FIGS. 6B and 6D, LEDs 66 (indicated by signs 66a and 66b in FIGS. 7A to 7C) inside the semitransparent soft cover 64 are caused to emit light, that is, the output set value up switch 62a and the output set value down switch 62b are caused to emit light, such that the light penetrates through the cover 64. Therefore, the shape of the various setting switches 30 stands out owing to the light. Further, different transparent materials are used for the semitransparent soft cover 64 to form a design, characters or the like. In this manner, the LEDs 66 inside the soft cover 64 penetrate light so that a shape such as the design or characters stands out, thereby allowing the various setting switches 30 to be more clearly indicated. In other words, the light emitting units 32a and 32b described in the first embodiment are replaced with the LEDs 66a and 66b (see FIGS. 7A to 7C) in this embodiment.

As shown in FIG. 4, the first handpiece 14a is connected to the handpiece connection portion 26 of the ultrasonic vibration driver 12. In a situation where the first handpiece 14a is connected, it is possible to change the output set value of the ultrasonic vibration driver 12.

Next, the function of the surgical operation system 10 according to this embodiment will be described.

The power switch 24 is switched on (the power switch 24 is turned on). In a situation where the power switch 24 is turned on, the output set value of the ultrasonic vibration driver 12 is at the minimum value. At this moment, since the output set value of the ultrasonic vibration driver 12 is at the minimum value, it is not possible to further decrease the output set value. Therefore, the output set value up switch 62a alone is operable of the output set value up switch 62a and the output set value down switch 62b. Thus, as shown in FIG. 7A, the LED 66a of the output set value up switch 62a is only lit (see FIGS. 6C and 6D), while the LED 66b of the output set value down switch 62b is continuously lit out (see FIGS. 6A and 6C). That is, the operable output set value up switch 62a only stands out and is recognized by the user.

From this state, the output set value up switch 62a of the ultrasonic vibration driver 12 is operated to increase the output set value. At this moment, the output set value can be decreased again or can be further increased. Therefore, both of the output set value up switch 62a and the output set value down switch 62b are operable. Thus, the LEDs 66a and 66b of both the output set value up switch 62a and the output set value down switch 62b are lit. That is, the output set value up switch 62a stays lit, while the output set value down switch 62b is newly lit. At this moment, both the output set value up switch 62a and the output set value down switch 62b stand out and are recognized by the user.

Furthermore, from this state, the output set value of the ultrasonic vibration driver 12 is increased to set the output set value at the maximum value. At this moment, since the output set value of the ultrasonic vibration driver 12 is at the maximum value, the output set value cannot be further increased. Therefore, the output set value down switch 62b alone is operable of the output set value up switch 62a and the output set value down switch 62b. Thus, the LED 66b of the output set value down switch 62b alone stays lit, while the LED 66a of the output set value up switch 62a is lit out. That is, the operable output set value down switch 62b only stands out and is recognized by the user.

When the first handpiece 14a is used, the switch which is lit is operated of the output set value up switch 62a and the output set value down switch 62b, thereby setting the output set value.

In this state, the handpiece 14 can be driven on the basis of the output set from the ultrasonic vibration driver 12. The user operates the foot switch 16 connected to the ultrasonic vibration driver 12 so that the handpiece 14 generates ultrasonic output oscillation to perform various treatments on a patient or the like.

Next, the power switch 24 is switched off. In this state, the first handpiece 14a is removed from the handpiece connection portion 26, and the second handpiece 14b is connected to the handpiece connection portion 26. At this moment, since the second handpiece 14b is connected to the handpiece connection portion 26 instead of the first handpiece 14a, the output set value of the ultrasonic vibration driver 12 cannot be changed. Therefore, in this state, the output set value up switch 62a and the output set value down switch 62b cannot be operated. Even if they are operated, such an operation cannot change the output set value up switch 62a and the output set value down switch 62b. Thus, the output set value up switch 62a and the output set value down switch 62b are continuously lit out. That is, both the output set value up switch 62a and the output set value down switch 62b cannot be visually recognized.

It is to be noted that the LED 66 of the operable switch is lit in the described embodiment, but LED 66 of the operable switch may flash.

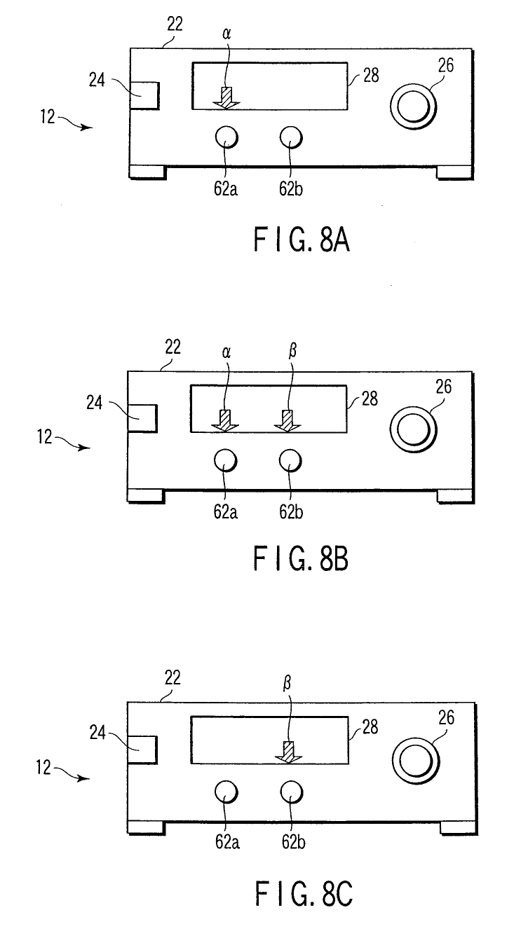

Next, a fifth embodiment will be described with FIGS. 8A to 8C. This embodiment is a modification of the first embodiment, so that the same signs are assigned to the same members as those described in the first embodiment and those members are not described in detail.

In this embodiment, as the setting switches 30 of the ultrasonic vibration driver 12, there are provided the output set value up switch 62a and the output set value down switch 62b. As shown in FIG. 4, the first handpiece 14a is connected to the handpiece connection portion 26 of the ultrasonic vibration driver 12. In a situation where the first handpiece 14a is connected, it is possible to change the output set value of the ultrasonic vibration driver 12.

It is to be noted that in a situation where the second handpiece 14b whose specification is different from that of the first handpiece 14a is connected to the handpiece connection portion 26, the control circuit 42 and the output circuit 48 of the ultrasonic vibration driver 12 are set so that the above second handpiece 14b cannot be used. Moreover, the display unit 28 is a liquid crystal display in the description.

In addition, the liquid crystal display is preferably capable of color display in that the display is easily recognized, but the liquid crystal display may be only capable of displaying in black and white. On the display unit 28, there is displayed an arrow, symbol, characters, picture or the like indicating the output set value up switch 62a. Here, arrows α, β indicate the operable switches.

Next, the function of the surgical operation system 10 according to this embodiment will be described.

The power switch 24 is switched on (the power switch 24 is turned on). In a situation where the power switch 24 is turned on, the output set value of the ultrasonic vibration driver 12 is at the minimum value. At this moment, since the output set value of the ultrasonic vibration driver 12 is at the minimum value, it is not possible to further decrease the output set value. Therefore, the output set value up switch 62a alone is operable of the output set value up switch 62a and the output set value down switch 62b. Thus, the operable switch is indicated by the arrow a which indicates the output set value up switch 62a in the display unit 28.

From this state, the output set value up switch 62a of the ultrasonic vibration driver 12 is operated to increase the output set value. At this moment, the output set value can be decreased again or can be further increased. Therefore, both of the output set value up switch 62a and the output set value down switch 62b is operable. Thus, both the output set value up switch 62a and the output set value down switch 62b are indicated by the arrows α, β in the display unit 28. That is, the output set value up switch 62a is continuously indicated by the arrow α, while the arrow β is newly displayed to indicate the output set value down switch 62b.

Furthermore, from this state, the output set value of the ultrasonic vibration driver 12 is increased to set the output set value at the maximum value. At this moment, since the output set value of the ultrasonic vibration driver 12 is at the maximum value, the output set value cannot be further increased. Therefore, the output set value down switch 62b alone is operable of the output set value up switch 62a and the output set value down switch 62b. Thus, the output set value down switch 62b alone is continuously indicated by the arrow β, while the display of the arrow α indicating the output set value up switch 62a disappears.

When the first handpiece 14a is used, the switch which is indicated by the arrow is operated of the output set value up switch 62a and the output set value down switch 62b, thereby setting the output set value.

In this state, the handpiece 14 can be driven on the basis of the output set from the ultrasonic vibration driver 12. The user operates the foot switch 16 connected to the ultrasonic vibration driver 12 so that the handpiece 14 generates ultrasonic output oscillation to perform various treatments on a patient or the like.

Next, the power switch 24 is switched off. In this state, the first handpiece 14a is removed from the handpiece connection portion 26, and the second handpiece 14b is connected to the handpiece connection portion 26. At this moment, since the second handpiece 14b is connected to the handpiece connection portion 26 instead of the first handpiece 14a, the output set value of the ultrasonic vibration driver 12 cannot be changed. Therefore, in this state, the output set value up switch 62a and the output set value down switch 62b cannot be operated. Even if they are operated, such an operation cannot change the output set value up switch 62a and the output set value down switch 62b. Thus, the output set value up switch 62a and the output set value down switch 62b are not indicated by the arrows α, β in the display unit 28.

It is to be noted that en example has been described here in which the switches 62a and 62b are provided side by side, but even if other switches are disposed on the right or left of the display unit 28, they can similarly be indicated by arrows or the like when they are operable.

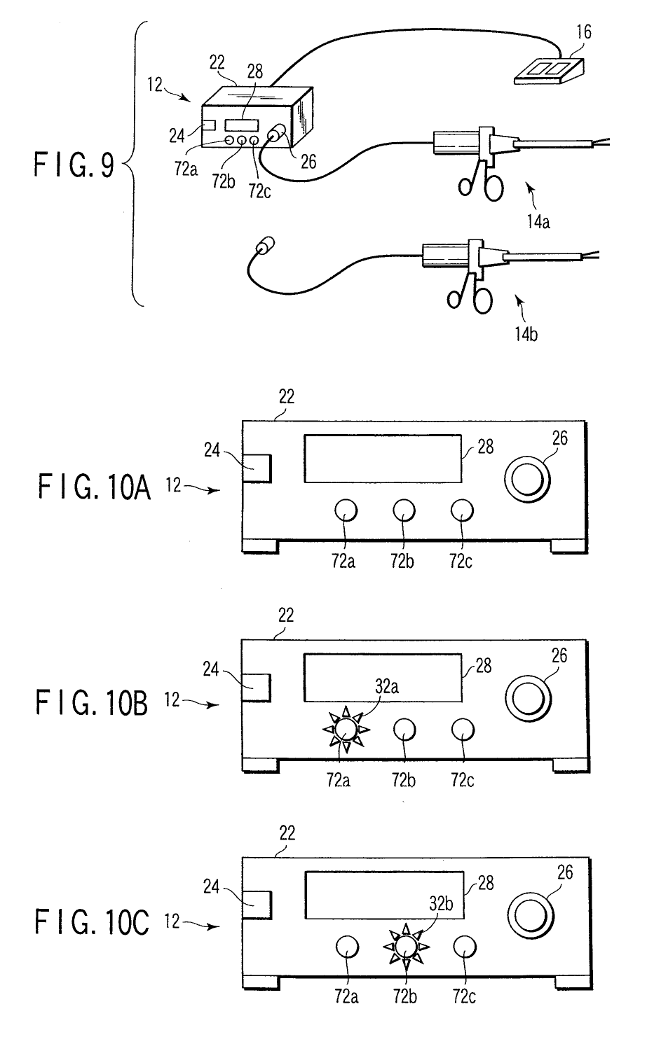

Next, a sixth embodiment will be described with FIGS. 9 to 10C. This embodiment is a modification of the first embodiment, so that the same signs are assigned to the same members as those described in the first embodiment and those members are not described in detail.

As shown in FIGS. 9 to 10C, the setting switches 30 of the ultrasonic vibration driver 12 according to this embodiment comprise a first output mode switch 72a, a second output mode switch 72b and a third output mode switch 72c. As shown in FIG. 9, the first handpiece 14a is connected to the handpiece connection portion 26 of the ultrasonic vibration driver 12. The first to third output mode switches 72a, 72b and 72c are capable of setting output states when a treatment using the first handpiece 14a is carried out. Thus, the first to third output mode switches 72a, 72b and 72c can be selected so that they are set in the same output mode, or they can also be set in different output modes (first to third output modes).

Next, the function of the surgical operation system 10 according to this embodiment will be described.

The power switch 24 is switched on (the power switch 24 is turned on). In a situation where the power switch 24 is turned on, the first to third output mode switches 72a, 72b and 72c are not pressed. Thus, as shown in FIG. 10A, the light emitting units 32a, 32b and 32b on the outer periphery of the first to third output mode switches 72a, 72b and 72c are lit out.

In this state, when the user selects to supply electric power from the ultrasonic vibration driver 12 to the handpiece 14 on the basis of the first output mode, the user presses the first output mode switch 72a. As shown in FIG. 10B, the first output mode switch 72a is lit for a given length of time or until the second and third output mode switches 72b and 72c are pressed. On the other hand, the second and third output mode switches 72b and 72c are continuously lit out.

In this state, the handpiece 14 can be driven on the basis of the output mode set in the ultrasonic vibration driver 12. The user operates the foot switch 16 connected to the ultrasonic vibration driver 12 so that the handpiece 14 generates ultrasonic output oscillation to perform various treatments on a patient or the like.

When the output mode of the handpiece 14 is changed to the second output mode, the second output mode switch 72b is selected. In this state, the user presses the second output mode switch 72b. Then, as shown in FIG. 10C, the second output mode switch 72b is lit, and the first output mode switch 72a is lit out. The second output mode switch 72b is lit for a given length of time or until the first and third output mode switches 72a and 72c are pressed. In addition, the third output mode switch 72c is continuously not lit.

When the output mode of the handpiece 14 is changed to the third output mode, the third output mode switch 72c is selected. In this case, the user presses the third output mode switch 72c. Then, the third output mode switch 72c is lit, and the second output mode switch 72b is lit out. The third output mode switch 72c is lit for a given length of time or until the first and second output mode switches 72a and 72b are pressed. In addition, the first output mode switch 72a is continuously lit out.

Next, a seventh embodiment will be described. This embodiment is a modification of the sixth embodiment, so that the same signs are assigned to the same members as those described in the sixth embodiment and those members are not described in detail.

In this embodiment, the first to third output mode switches 72a, 72b and 72c of the ultrasonic vibration driver 12 are provided with indication lights which change the color of emitted light.

Next, the function of the surgical operation system 10 according to this embodiment will be described.

The power switch 24 is switched on (the power switch 24 is turned on). At this moment, the first to third output mode switches 72a, 72b and 72c always keep lighting.

In this state, when the user selects to supply electric power from the ultrasonic vibration driver 12 to the handpiece 14 on the basis of the first output mode, the user presses the first output mode switch 72a. The color of the light emitted by the first output mode switch 72a changes, for example, until the other second and third output mode switches 72b and 72c are pressed. On the other hand, the second and third output mode switches 72b and 72c keep lighting as in the state exhibited when the power switch 24 is turned on.

In this state, the handpiece 14 can be driven on the basis of the output mode set in the ultrasonic vibration driver 12. The user operates the foot switch 16 connected to the ultrasonic vibration driver 12 so that the handpiece 14 generates ultrasonic output oscillation to perform various treatments on a patient or the like.

When the output mode of the handpiece 14 is changed to the second output mode, the second output mode switch 72b is selected. In this case, the user presses the second output mode switch 72b. Then, the color of the light emitted by the second output mode switch 72b changes, and the color of the light emitted by the first output mode switch 72a returns to the state exhibited when the power switch 24 is turned on. The second output mode switch 72b maintains the changing state of the emitted light for a given length of time or until the other first and third switches 72a and 72c are pressed. It is to be noted that the third output mode switch 72c stays lit as in the state exhibited when the power switch 24 is turned on.

When the output mode of the handpiece 14 is changed to the third output mode, the third output mode switch 72c is selected. In this case, the user presses the third output mode switch 72c. Then, the color of the light emitted by the third output mode switch 72c changes, and the color of the light emitted by the second output mode switch 72b returns to the state exhibited when the power switch 24 is turned on. The third output mode switch 72c maintains the changing state of the emitted light for a given length of time or until the other first and second switches 72a and 72b are pressed. It is to be noted that the first output mode switch 72a stays lit as in the state exhibited when the power switch 24 is turned on.

Next, an eighth embodiment will be described. This embodiment is a modification of the sixth and seventh embodiments, so that the same signs are assigned to the same members as those described in the sixth and seventh embodiments and those members are not described in detail.

In this embodiment, the first to third output mode switches 72a, 72b and 72c of the ultrasonic vibration driver 12 are provided with indication lights which are in a lighting state when the power switch 24 is turned on and which generate colored lights and flash.

Next, the function of the surgical operation system 10 according to this embodiment will be described.

The power switch 24 is switched on (the power switch 24 is turned on). At this moment, the first to third output mode switches 72a, 72b and 72c always keep lighting.

In this state, when the user selects to supply electric power from the ultrasonic vibration driver 12 to the handpiece 14 on the basis of the first output mode, the user presses the first output mode switch 72a. The first output mode switch 72a changes the color of the emitted light and flashes, for example, until the other second and third output mode switches 72b and 72c are pressed. On the other hand, the second and third output mode switches 72b and 72c keep lighting as in the state exhibited when the power switch 24 is turned on.

In this state, the handpiece 14 can be driven on the basis of the output mode set in the ultrasonic vibration driver 12. The user operates the foot switch 16 connected to the ultrasonic vibration driver 12 so that the handpiece 14 generates ultrasonic output oscillation to perform various treatments on a patient or the like.

When the output mode of the handpiece 14 is changed to the second output mode, the second output mode switch 72b is selected. In this case, the user presses the second output mode switch 72b. Then, the second output mode switch 72b changes the color of the emitted light and flashes, and the first output mode switch 72a stops flashing the colored lights and returns to the state exhibited when the power switch 24 is turned on. The second output mode switch 72b maintains the color-changing and flashing state of the emitted light for a given length of time or until the other first and third switches 72a and 72c are pressed. It is to be noted that the third output mode switch 72c stays lit as in the state exhibited when the power switch 24 is turned on.

When the output mode of the handpiece 14 is changed to the third output mode, the third output mode switch 72c is selected. In this case, the user presses the third output mode switch 72c. Then, the third output mode switch 72c changes the color of the emitted light and flashes, and the second output mode switch 72b stops flashing the colored lights and returns to the state exhibited when the power switch 24 is turned on. The third output mode switch 72c maintains the color-changing and flashing state of the emitted light for a given length of time or until the other first and second switches 72a and 72b are pressed. It is to be noted that the first output mode switch 72a stays lit as in the state exhibited when the power switch 24 is turned on.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general invention concept as defined by the appended claims and their equivalents.

What is claimed is:

1. A surgical operation apparatus comprising:
a main body;
a plurality of switches which are provided in the main body and configured to be operated by a user;
a plurality of light emitting units which are provided in the switches themselves or on the periphery of the switches and which light the switches; and
a control section which is configured to determine whether or not each target controlled by the plurality of switches is suitable for being controlled by the plurality of switches and the control section controls the light emitting unit of a switch, corresponding to a controllable target among the plurality of switches, to emit light.
